(19) United States
(12) Patent Application Publication
(10) Pub. No.: US 2024/0374607 A1
(43) Pub. Date: Nov. 14, 2024

(54) MEDICAMENT FOR PREVENTING AND/OR TREATING DRY EYE

(71) Applicants: Santen Pharmaceutical Co., Ltd., Osaka-shi, Osaka (JP); Boehringer Ingelheim International GMBH, Ingelheim am Rhein (DE)

(72) Inventors: Kenji Oki, Minoh-shi, Osaka (JP); Takashi Nagano, Osaka-shi, Osaka (JP); Nagayoshi Asano, Ikoma-shi, Nara (JP); Koushi Fujisawa, Ikoma-shi, Nara (JP)

(21) Appl. No.: 18/691,241

(22) PCT Filed: Sep. 14, 2022
(86) PCT No.: PCT/JP2022/034383

§ 371 Date: Mar. 12, 2024

(30) Foreign Application Priority Data
Sep. 15, 2021 (JP) 2021-150112

(51) Int. Cl. A61K 31/538; A61P 27/04

(52) U.S. Cl. CPC A61K 31/538; A61P 27/04

(57) ABSTRACT

A compound useful for preventing or treating dry eye and a medicament for preventing and/or treating dry eye comprising the same which does not substantially impact on the intraocular pressure are provided. The present invention relates to a medicament for preventing and/or treating dry eye, comprising olodaterol or a salt thereof as an active ingredient.

### TECHNICAL FIELD

The present invention relates to a medicament for preventing and/or treating dry eye, comprising olodaterol or a salt thereof as an active ingredient.

### BACKGROUND ART

Dry eye is a disease causing dry-eye symptoms such as discomfort feelings on eyes and abnormalities in visual performance, associated with abnormalities in tear volume or quality, which may cause disorders on the surface of eyes such as cornea. The abnormalities in tear volume mainly refer to the condition of low lacrimal secretion, which is an index of dry eye based on the tear volume. Regarding the abnormalities in tear quality, abnormalities in tear ingredients such as few lipid or protein components contained in tears may deteriorate the stability of tear film, which may then cause dryness on the surface of eyes even with tear secretion.

Clenbuterol and fenoterol are known as a selective β₂ adrenergic receptor agonist, and have been confirmed to enhance secretion of tears and tear proteins (Patent Literature 1).

Another β₂ adrenergic receptor agonist, salbutamol, has also been confirmed to enhance lacrimal secretion (Patent Literature 2). Whereas, it has been reported that salbutamol did not have sufficient effects on the treatment of dry eye, and increased intraocular pressure (Patent Literature 3).

Patent Literature 4 lists dry eye as an example of various symptoms associated with autoimmune disease, systemic lupus erythematosus (SLE), and discloses the use of β₂ adrenergic receptor agonist in the treatment of SLE. It discloses tests with some drugs in a kidney disease model, but it does not disclose any tests for the treatment of dry eye with β₂ adrenergic receptor agonists.

Olodaterol (chemical name: 6-hydroxy-8-((1R)-1-hydroxy-2-{[2-(4-methoxyphenyl)-1,1-dimethylethyl]amino}&sh y;ethyl)-2H-1,4-benzoxazin-3-(4H)-one) is known as a selective β₂ adrenergic receptor agonist, which is represented by the following formula:

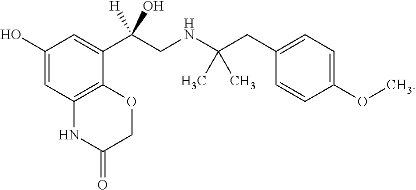 [SMILES: COc1ccc(CC(C)(C)NC[C@H](O)c2cc(O)cc3c2OCC(=O)N3)cc1]

Since β₂ adrenergic receptors are generally present in the bronchi or blood vessels, olodaterol hydrochloride has been launched as a drug for treating chronic obstructive pulmonary disease (COPD) in the form of a combination drug with tiotropium. Effects of olodaterol on the treatment of eye diseases, particularly on dry eye, have not been known so far.

A β₂ adrenergic receptor antagonist, timolol, is used for a medicament for treating glaucoma or ocular hypertension, and has ocular hypotensive effects. In particular, it is desirable that a β₂ adrenergic receptor agonist as a medicament for treating dry eye does not substantially impact on the intraocular pressure; i.e., it does not cause side effects such as the increase or reduction of intraocular pressure.

### CITATION LIST

### Patent Literature

[PL 1] WO 01/41806

[PL 2] WO 2011/068786

[PL 3] WO 2017/043612

[PL 4] WO 2019/020634

### SUMMARY OF INVENTION

### Technical Problem

A problem to be solved by the present invention is to provide a compound useful for preventing or treating dry eye and a medicament for preventing and/or treating dry eye comprising the same which does not substantially impact on intraocular pressure.

### Solution to Problem

The present inventors have extensively studied to solve the above problem, and then surprisingly have found that olodaterol or a salt thereof stabilizes tear film, enhances lacrimal secretion, and/or increases tear volumes, which may prevent and/or treat dry eye without a substantial impact on intraocular pressure, and have achieved the invention.

In one embodiment, a medicament for preventing and/or treating dry eye, comprising olodaterol or a salt thereof as an active ingredient, is provided. In another embodiment, an agent for stabilizing tear film, comprising olodaterol or a salt thereof as an active ingredient, is provided. In still another embodiment, an agent for enhancing lacrimal secretion and/or increasing tear volumes, comprising olodaterol or a salt thereof as an active ingredient, is provided.

### Advantageous Effects of Invention

Olodaterol or a salt thereof may extend the tear film break-up time to stabilize tear film in a dose-dependent manner, and is expected to be useful for prevention and/or treatment of dry eye. Olodaterol or a salt thereof may also enhance tear secretion and increase tear volumes, and may significantly increase tear volumes even in lower concentrations than do other selective β₂ adrenergic receptor agonists, which allows for the provision of effects on prevention and/or treatment of dry eye with lower exposure. The provision of effects on prevention and/or treatment with low exposure may decrease risks of systemic and/or local side effects. No substantial increase or reduction of intraocular pressure during a prescribed period after administration allows for the provision of a medicament of prevention and/or treatment with high safety to eyes.

### BRIEF DESCRIPTION OF DRAWINGS

FIG. 1 shows the effect of extending tear film break-up time and time-course changes thereof at each concentration after a single ocular instillation of 0.0001% (w/v), 0.001% (w/v), or 0.01% (w/v) of olodaterol to cynomolgus monkeys.

FIG. 2 shows the effect of extending tear film break-up time and time-course changes thereof after a single ocular instillation of 1% (w/v) fenoterol or 1% (w/v) clenbuterol ophthalmic solution to cynomolgus monkeys.

FIG. 3 shows the effect of increasing tear volumes and time-course changes thereof at each concentration after a single ocular instillation of 0.00001% (w/v), 0.0001% (w/v), 0.001% (w/v), or 0.01% (w/v) of olodaterol to rats.

FIG. 4 shows temporal effects on the intraocular pressure in twice daily repeated administration of 0.01% (w/v) or 0.1% (w/v) of olodaterol ophthalmic solution to cynomolgus monkeys.

FIG. 5 shows the effect of extending tear film break-up time and time-course changes thereof after a single ocular instillation of 0.00001% (w/v), 0.0001 (w/v), 0.001 (w/v), or 0.01% (w/v) of olodaterol to SD rats.

### DESCRIPTION OF EMBODIMENTS

Several embodiments are illustrated as follows.

[Item 1] A medicament for preventing and/or treating dry eye, comprising olodaterol or a salt thereof as an active ingredient.

[Item 2] The medicament according to Item 1, wherein the dry eye accompanies corneal and conjunctival epithelium disorder caused by at least one selected from the group consisting of superficial punctate keratopathy, corneal epithelium defect, corneal erosion, corneal ulcer, conjunctival epithelium defect, keratoconjunctivitis sicca, superior limbic keratoconjunctivitis, filamentary keratoconjunctivitis, non-infectious keratitis, and non-infectious conjunctivitis.

[Item 3] The medicament according to Item 1, wherein the dry eye is a disease caused by at least one selected from the group consisting of tear film instability, lacrimal hyposecretion, xerophthalmia, tear deficiency, keratoconjunctivitis sicca, lacrimal gland dysfunction, meibomian gland dysfunction, VDT (Visual Display Terminal) operation, use of contact lenses, tear abnormalities, and keratoconjunctivitis.

[Item 4] The medicament according to any one of Items 1 to 3, wherein the olodaterol or a salt thereof is comprised in a concentration of 0.1% (w/v) or less.

[Item 5] The medicament according to any one of Items 1 to 3, wherein the olodaterol or a salt thereof is comprised in a concentration of 0.01% (w/v) or less.

[Item 6] The medicament according to any one of Items 1 to 3, wherein the olodaterol or a salt thereof is comprised in a concentration of 0.00001 to 0.1% (w/v).

[Item 7] The medicament according to any one of Items 1 to 3, wherein the olodaterol or a salt thereof is comprised in a concentration of 0.00001 to 0.01% (w/v).

[Item 8] The medicament according to any one of Items 1 to 3, wherein the olodaterol or a salt thereof is comprised in a concentration of 0.00001 to 0.005% (w/v).

[Item 9] The medicament according to any one of Items 1 to 3, wherein the olodaterol or a salt thereof is comprised in a concentration of 0.00001 to 0.001% (w/v).

[Item 10] The medicament according to any one of Items 1 to 9, wherein the medicament is in the form of an eye drop.

[Item 11] The medicament according to any one of Items 1 to 10, wherein the medicament does not substantially increase and/or reduce intraocular pressure.

[Item 12] The medicament according to any one of Items 1 to 11, wherein the olodaterol or a salt thereof is olodaterol hydrochloride.

[Item 13] An agent for stabilizing tear film, comprising olodaterol or a salt thereof as an active ingredient.

[Item 14] An agent for enhancing lacrimal secretion and/or increasing tear volumes, comprising olodaterol or a salt thereof as an active ingredient.

[Item 15] A method of preventing and/or treating dry eye, comprising administering a therapeutically effective amount of olodaterol or a salt thereof to a subject.

[Item 16] The method according to Item 15, wherein the dry eye accompanies corneal and conjunctival epithelium disorder caused by at least one selected from the group consisting of superficial punctate keratopathy, corneal epithelium defect, corneal erosion, corneal ulcer, conjunctival epithelium defect, keratoconjunctivitis sicca, superior limbic keratoconjunctivitis, filamentary keratoconjunctivitis, non-infectious keratitis, and non-infectious conjunctivitis.

[Item 17] The medicament according to Item 15, wherein the dry eye is a disease caused by at least one selected from the group consisting of tear film instability, lacrimal hyposecretion, xerophthalmia, tear deficiency, keratoconjunctivitis sicca, lacrimal gland dysfunction, meibomian gland dysfunction, VDT (Visual Display Terminal) operation, use of contact lenses, tear abnormalities, and keratoconjunctivitis.

[Item 18] The method according to any one of Items 15 to 17, wherein the concentration of the olodaterol or a salt thereof is 0.1% (w/v) or less.

[Item 19] The method according to any one of Items 15 to 17, wherein the concentration of the olodaterol or a salt thereof is 0.01% (w/v) or less.

[Item 20] The method according to any one of Items 15 to 17, wherein the concentration of the olodaterol or a salt thereof is 0.00001 to 0.1% (w/v).

[Item 21] The method according to any one of Items 15 to 17, wherein the concentration of the olodaterol or a salt thereof is 0.00001 to 0.01% (w/v).

[Item 22] The method according to any one of Items 15 to 17, wherein the concentration of the olodaterol or a salt thereof is 0.00001 to 0.005% (w/v).

[Item 23] The method according to any one of Items 15 to 17, wherein the concentration of the olodaterol or a salt thereof is 0.00001 to 0.001% (w/v).

[Item 24] The method according to any one of Items 15 to 23, wherein the olodaterol or a salt thereof is administered in the form of an eye drop.

[Item 25] The method according to any one of Items 15 to 24, which does not substantially increase and/or reduce intraocular pressure.

[Item 26] The method according to any one of Items 15 to 25, wherein the olodaterol or a salt thereof is olodaterol hydrochloride.

[Item 27] A method of stabilizing tear film, comprising administering a therapeutically effective amount of olodaterol or a salt thereof to a subject.

[Item 28] A method of enhancing lacrimal secretion and/or increasing tear volumes, comprising administering a therapeutically effective amount of olodaterol or a salt thereof to a subject.

[Item 29] Olodaterol or a salt thereof for use in preventing and/or treating dry eye.

[Item 30] The olodaterol or a salt thereof according to Item 29, wherein the dry eye accompanies corneal and conjunctival epithelium disorder caused by at least one selected from the group consisting of superficial punctate keratopathy, corneal epithelium defect, corneal erosion, corneal ulcer, conjunctival epithelium defect, keratoconjunctivitis sicca, superior limbic keratoconjunctivitis, filamentary keratoconjunctivitis, non-infectious keratitis, and non-infectious conjunctivitis.

[Item 31] The olodaterol or a salt thereof according to Item 29, wherein the dry eye is a disease caused by at least one selected from the group consisting of tear film instability, lacrimal hyposecretion, xerophthalmia, tear deficiency, keratoconjunctivitis sicca, lacrimal gland dysfunction, meibomian gland dysfunction, VDT (Visual Display Terminal) operation, use of contact lenses, tear abnormalities, and keratoconjunctivitis.

[Item 32] The olodaterol or a salt thereof according to any one of Items 29 to 31 in a concentration of 0.1% (w/v) or less.

[Item 33] The olodaterol or a salt thereof according to any one of Items 29 to 31 in a concentration of 0.01% (w/v) or less.

[Item 34] The olodaterol or a salt thereof according to any one of Items 29 to 31 in a concentration of 0.00001 to 0.1% (w/v).

[Item 35] The olodaterol or a salt thereof according to any one of Items 29 to 31 in a concentration of 0.00001 to 0.01% (w/v).

[Item 36] The olodaterol or a salt thereof according to any one of Items 29 to 31 in a concentration of 0.00001 to 0.005% (w/v).

[Item 37] The olodaterol or a salt thereof according to any one of Items 29 to 31 in a concentration of 0.00001 to 0.001% (w/v).

[Item 38] The olodaterol or a salt thereof according to any one of Items 29 to 37 in the form of an eye drop.

[Item 39] The olodaterol or a salt thereof according to any one of Items 29 to 38, which does not substantially increase and/or reduce intraocular pressure.

[Item 40] The olodaterol or a salt thereof according to any one of Items 29 to 39 which is olodaterol hydrochloride.

[Item 41] Olodaterol or a salt thereof for use in stabilizing tear film.

[Item 42] Olodaterol or a salt thereof for use in enhancing lacrimal secretion and/or increasing tear volumes.

[Item 43] Use of olodaterol or a salt thereof in the manufacture of a medicament for preventing and/or treating dry eye.

[Item 44] The use according to Item 43, wherein the dry eye accompanies corneal and conjunctival epithelium disorder caused by at least one selected from the group consisting of superficial punctate keratopathy, corneal epithelium defect, corneal erosion, corneal ulcer, conjunctival epithelium defect, keratoconjunctivitis sicca, superior limbic keratoconjunctivitis, filamentary keratoconjunctivitis, non-infectious keratitis, and non-infectious conjunctivitis.

[Item 45] The use according to Item 43, wherein the dry eye is a disease caused by at least one selected from the group consisting of tear film instability, lacrimal hyposecretion, xerophthalmia, tear deficiency, keratoconjunctivitis sicca, lacrimal gland dysfunction, meibomian gland dysfunction, VDT (Visual Display Terminal) operation, use of contact lenses, tear abnormalities, and keratoconjunctivitis.

[Item 46] The use according to any one of Items 43 to 45, wherein the concentration of the olodaterol or a salt thereof is 0.1% (w/v) or less.

[Item 47] The use according to any one of Items 43 to 45, wherein the concentration of the olodaterol or a salt thereof is 0.01% (w/v) or less.

[Item 48] The use according to any one of Items 43 to 45, wherein the concentration of the olodaterol or a salt thereof is 0.00001 to 0.1% (w/v).

[Item 49] The use according to any one of Items 43 to 45, wherein the concentration of the olodaterol or a salt thereof is 0.00001 to 0.01% (w/v).

[Item 50] The use according to any one of Items 43 to 45, wherein the concentration of the olodaterol or a salt thereof is 0.00001 to 0.005% (w/v).

[Item 51] The use according to any one of Items 43 to 45, wherein the concentration of the olodaterol or a salt thereof is 0.00001 to 0.001% (w/v).

[Item 52] The use according to any one of Items 43 to 51, wherein the medicament is in the form of an eye drop.

[Item 53] The use according to any one of Items 43 to 52, wherein the medicament does not substantially increase and/or reduce intraocular pressure.

[Item 54] The use according to any one of Items 43 to 53, wherein the olodaterol or a salt thereof is olodaterol hydrochloride.

[Item 55] Use of olodaterol or a salt thereof in the manufacture of a medicament for stabilizing tear film.

[Item 56] Use of olodaterol or a salt thereof in the manufacture of a medicament for enhancing lacrimal secretion and/or increasing tear volumes.

Olodaterol is a selective agonist for human β₂ adrenergic receptor, represented by a general formula as below, which shows a weak intrinsic activity against β₁ adrenergic receptors, while showing almost perfect intrinsic activity against β₂ adrenergic receptors and having high β₂ adrenergic receptor selectivity.

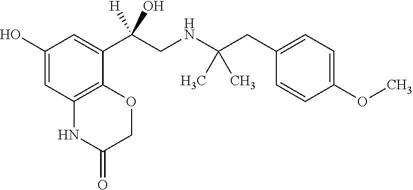

The term “olodaterol or a salt thereof” used herein includes olodaterol and its salt with inorganic or organic acid. Such a salt of olodaterol includes, for example, hydrochloride, hydrosulfate, acetate, trifluoroacetate, hydrobromide, hydroiodide, hydrophosphate, hydromethanesulfonate, hydronitrate, hydromaleate, hydroacetate, hydrobenzoate, hydrocitrate, hydrofumarate, hydrotartrate, hydrooxalate, hydrosuccinate, hydrobenzoate, and hydro-p-toluenesulfonate; preferably, hydrochloride, hydrobromide, hydrosulfate, hydrophosphate, hydrofumarate, and hydromethanesulfonate; more preferably, olodaterol hydrochloride. Salts can be prepared according to conventional methods in the field of organic synthetic chemistry or the methods described in, for example, WO 2004/045618 and US 2007/0027148 A.

Olodaterol or a salt thereof may be present in the form of a hydrate or a solvate. Such a solvate includes, for example, ethanolate. A hydrate and a solvate can be prepared according to conventional methods in the field of organic synthetic chemistry or may be commercially available.

When olodaterol or a salt thereof has crystal polymorphs and crystal polymorph group (i.e., crystal polymorph system), such crystal polymorphs and crystal polymorph group (i.e., crystal polymorph system) may also be encompassed in the scope of the present invention. When crystalline forms vary depending on conditions and states of preparation, crystallization, and storage of crystals, the crystal polymorph group (i.e., crystal polymorph system) includes not only each crystalline form obtained in each of these steps, but also any mixtures of crystalline forms obtained in two or more of these steps.

Herein, the concentration of olodaterol or a salt thereof in a medicament is not limited, but is preferably 0.1% (w/v) or less and/or below, more preferably 0.01% (w/v) or less and/or below, still more preferably 0.005% (w/v) or less and/or below. The upper limit of the concentration includes 0.1% (w/v), 0.05% (w/v), 0.03% (w/v), 0.01% (w/v), 0.005% (w/v), 0.003% (w/v), and 0.001% (w/v). The lower limit of the concentration includes 0.0000001% (w/v), 0.000001% (w/v), 0.000003% (w/v), 0.000005% (w/v), 0.00001% (w/v), 0.00003% (w/v), 0.00005% (w/v), 0.0001% (w/v), 0.0003% (w/v), and 0.0005% (w/v). The range of the concentrations includes 0.0000001 to 0.1% (w/v), 0.0000001 to 0.05% (w/v), 0.0000001 to 0.03% (w/v), 0.0000001 to 0.01% (w/v), 0.0000001 to 0.005% (w/v), 0.0000001 to 0.003% (w/v), 0.0000001 to 0.001% (w/v), 0.000001 to 0.1% (w/v), 0.000001 to 0.05% (w/v), 0.000001 to 0.03% (w/v), 0.000001 to 0.01% (w/v), 0.000001 to 0.005% (w/v), 0.000001 to 0.003% (w/v), 0.000001 to 0.001% (w/v), 0.000003 to 0.1% (w/v), 0.000003 to 0.05% (w/v), 0.000003 to 0.03% (w/v), 0.000003 to 0.01% (w/v), 0.000003 to 0.005% (w/v), 0.000003 to 0.003% (w/v), 0.000003 to 0.001% (w/v), 0.000005 to 0.1% (w/v), 0.000005 to 0.05% (w/v), 0.000005 to 0.03% (w/v), 0.000005 to 0.01% (w/v), 0.000005 to 0.005% (w/v), 0.000005 to 0.003% (w/v), 0.000005 to 0.001% (w/v), 0.00001 to 0.1% (w/v), 0.00001 to 0.05% (w/v), 0.00001 to 0.03% (w/v), 0.00001 to 0.01% (w/v), 0.00001 to 0.005% (w/v), 0.00001 to 0.003% (w/v), 0.00001 to 0.001% (w/v), 0.00003 to 0.1% (w/v), 0.00003 to 0.05% (w/v), 0.00003 to 0.03% (w/v), 0.00003 to 0.01% (w/v), 0.00003 to 0.005% (w/v), 0.00003 to 0.003% (w/v), 0.00003 to 0.001% (w/v), 0.00005 to 0.1% (w/v), 0.00005 to 0.05% (w/v), 0.00005 to 0.03% (w/v), 0.00005 to 0.01% (w/v), 0.00005 to 0.005% (w/v), 0.00005 to 0.003% (w/v), 0.00005 to 0.001% (w/v), 0.0001 to 0.1% (w/v), 0.0001 to 0.05% (w/v), 0.0001 to 0.03% (w/v), 0.0001 to 0.01% (w/v), 0.0001 to 0.005% (w/v), 0.0001 to 0.003% (w/v), 0.0001 to 0.001% (w/v), 0.0003 to 0.1% (w/v), 0.0003 to 0.05% (w/v), 0.0003 to 0.03% (w/v), 0.0003 to 0.01% (w/v), 0.0003 to 0.005% (w/v), 0.0003 to 0.003% (w/v), 0.0003 to 0.001% (w/v), 0.0005 to 0.1% (w/v), 0.0005 to 0.05% (w/v), 0.0005 to 0.03% (w/v), 0.0005 to 0.01% (w/v), 0.0005 to 0.005% (w/v), 0.0005 to 0.003% (w/v), and 0.0005 to 0.001% (w/v).

β₂ adrenergic receptor agonists are known to cause systemic side effects such as cardiovascular side effects (e.g., increase of heart rate and arrhythmia) when also acting on β₁ subtypes, many of which are present in the heart (Patent Literature 1). Olodaterol or a salt thereof does not substantially show systemic and/or local side effects in any of the above concentrations or less than these concentrations, and is expected to provide a medicament with good tolerability.

Olodaterol or a salt thereof may be administered by formulating into a medicament form comprising the same as an active ingredient. Such a form for administration includes, for example, the form of parenteral administration, preferably the form of ocular instillation. A dosage form includes, for example, an ophthalmic composition. The ophthalmic composition may be a medicament with prophylactic or therapeutic effects, that is, the effects of alleviating, improving, relieving, inhibiting progression of, preventing, or delaying eye diseases and the symptoms thereof in a subject or patient, and includes, for example, an eye drop and an eye ointment, preferably an eye drop, more preferably an aqueous eye drop. The ophthalmic composition may be a medicament prescribed by a doctor or a medicament commonly sold in pharmacies, etc. (e.g., artificial tears, lubricants).

Herein, a medicament may be prepared by formulating an active ingredient with pharmaceutically acceptable carriers. Such pharmaceutically acceptable carriers include, for example, buffering agents, tonicity agents, pH adjusters, preserving agents, stabilizing agents, and vehicles. The amount of the carriers contained in a medicament is not limited, and may be any amounts commonly used in the field.

Dosage, also herein referred to as a “therapeutically effective amount”, of a medicament herein is not limited as long as it provides a desired therapeutic efficacy and tolerability. For example, it is preferable to instill 1 to 3 drops at a time per one eye for an eye drop, more preferably 1 or 2 drops at a time, still more preferably 1 drop at a time. It is preferable to instill 1 to 6 times daily per one eye, more preferably 1 to 3 times daily, still more preferably 1 or 2 times daily, particularly preferably once daily. It is further preferable to instill 1 drop at a time 1 or 2 times daily per one eye, more preferably 1 drop at a time once daily.

The term “dry eye” used herein is a disease causing dry-eye symptoms such as discomfort feelings on eyes and reduction or abnormalities in visual performance, associated with abnormalities in tear volume or quality, which may cause disorders on the surface of eyes such as cornea. Such dry-eye symptoms include, for example, eye fatigue, itchy eyes, eye pain, tearing, discharge, dryness of eyes, blurred vision, eye redness, eye irritation, feeling of having something in eyes (rumbling or tingling sensation in eyes), discomfort feelings on eyes when wearing soft contact lenses or hard contact lenses, and burning sensation, inflammation and discomfort feelings on eyes including the exposure of eyes to wind and sun. Dry eye may be associated with and/or caused by at least one selected from the group consisting of corneal and conjunctival epithelium disorder caused by at least one selected from the group consisting of superficial punctate keratopathy, corneal epithelium defect, corneal erosion, corneal ulcer, conjunctival epithelium defect, keratoconjunctivitis sicca, superior limbic keratoconjunctivitis, filamentary keratoconjunctivitis, non-infectious keratitis, and non-infectious conjunctivitis, tear film instability, lacrimal hyposecretion, xerophthalmia, tear deficiency, Sjogren syndrome, keratoconjunctivitis sicca, Stevens-Johnson syndrome, Graves' disease, lacrimal gland dysfunction, meibomian gland dysfunction, diabetes, graft-versus-host disease, VDT (Visual Display Terminal) operation, surgery, medicine, trauma, use of contact lenses, tear abnormalities, and keratoconjunctivitis. Preferably, dry eye may be associated with and/or caused by at least one selected from the group consisting of corneal and conjunctival epithelium disorder caused by at least one selected from the group consisting of superficial punctate keratopathy, corneal epithelium defect, corneal erosion, corneal ulcer, conjunctival epithelium defect, keratoconjunctivitis sicca, superior limbic keratoconjunctivitis, filamentary keratoconjunctivitis, non-infectious keratitis, and non-infectious conjunctivitis, tear film instability, lacrimal hyposecretion, xerophthalmia, tear deficiency, keratoconjunctivitis sicca, lacrimal gland dysfunction, meibomian gland dysfunction, VDT (Visual Display Terminal) operation, use of contact lenses, tear abnormalities, and keratoconjunctivitis.

Herein, the effect of stabilizing tear film is mainly associated with abnormalities in tear quality, and is the effect of extending the tear film break-up time (also herein referred to as “TBUT”) which is an index of the tear film stability covering the surface of cornea, which can be assessed by measuring TBUT. Specifically, when the time until tear film covering cornea is disrupted after eyes open unblinkingly is measured, then it may be diagnosed for human beings as normal when the time is 10 seconds or over, and diagnosed as dry eye when the time is 5 seconds or less. TBUT is usually measured with a staining solution such as fluorescein, but the measurement is not limited thereto. More specifically, it can be assessed, for example, under the condition of Test 1 below. When dry eye is associated with tear film instability, dry eye may be short tear film break-up time type of dry eye. A medicament herein may extend TBUT, for example, even at 15 minutes, preferably 30 minutes, more preferably 60 minutes, after administration. The extending effect may depend on the dose of an active ingredient. Other selective β₂ adrenergic receptor agonists did not have the TBUT-extending effect as shown in Test 2 below, whereas olodaterol showed the sustained TBUT-extending effect even in low concentrations as shown in Test 1. The effect of olodaterol on stabilizing tear film is considered to be extraordinary, unlike other β₂ adrenergic receptor agonists.

Herein, the effect of increasing tear volumes is the effect of improving tear volumes and/or tear quality, which enhances the secretion of tears and/or tear proteins. The effect of increasing tear volumes can be assessed by measuring the amount of lacrimal secretion under, for example, Schirmer's test. Specifically, it can be assessed under, for example, the condition of Test 3 below. When dry eye is associated with a decrease in the amount of lacrimal secretion, dry eye may be aqueous deficient dry eye, and may be associated with Sjogren syndrome. When dry eye is associated with abnormalities in tear quality, dry eye may be evaporative dry eye, and may be associated with meibomian gland dysfunction. The effect of increasing tear volumes may be elicited even by olodaterol in low concentrations, for example, 0.1% (w/v) or less, 0.01% (w/v) or less, 0.001% (w/v) or less, and 0.0001% (w/v) or less. The effect of increasing tear volumes may be sustained for, for example, 0.5 hours or more, 2 hours or more, 8 hours or more, and 16 hours or more, after ocular instillation.

The phrase “does not substantially increase and/or reduce intraocular pressure” used herein means the case where no significant difference in intraocular pressure is produced compared to a control in a single administration or repeated administration of an active ingredient to a subject for a certain period. In one embodiment, the phrase “does not substantially increase and/or reduce intraocular pressure” means the case where no significant difference in intraocular pressure is produced compared to a control in once-daily administration or two- to six-times-daily repeated administration of 0.1% (w/v) or less of an active ingredient to a subject, for example, immediately after administration, or at 2 hours, 4 hours, 2 days, 7 days, or 14 days, after administration, and the intraocular pressure can be assessed under, for example, the condition of Test 5 below.

The term “preventing” used herein includes the prevention and delay of the onset of dry eye symptoms. Olodaterol or a salt thereof may stabilize tear film and increase tear volumes, and is expected to be useful for the prevention of dry eye.

The term “treating” used herein includes the alleviation, improvement, and remission of dry eye symptoms, and the inhibition, prevention, and delay of progression.

### EXAMPLES

The invention is illustrated in more detail by Examples and Tests below, but is not intended to be limited thereto. Concentrations refer to weight to volume % “w/v (g/100 mL) %”, unless otherwise specified.

### [Test 1] Effect of Extending Tear Film Break-Up Time

The effect of olodaterol ophthalmic solutions on extension of tear film break-up time was studied.

### Sample Preparation

The following ophthalmic solution 1 was prepared as an olodaterol ophthalmic solution for use in the test.

### Ophthalmic Solution 1:

Olodaterol hydrochloride (0.0001 g), a buffering agent, and a tonicity agent were dissolved in water so as to be 100 mL, and a pH adjuster was added to the solution to prepare an olodaterol ophthalmic solution in the concentration of 0.0001% (w/v).

Olodaterol ophthalmic solutions in the concentrations of 0.001 and 0.01% (w/v) were also prepared with varied amounts of olodaterol hydrochloride.

An ophthalmic solution without containing olodaterol (vehicle) was prepared as a control group for the olodaterol ophthalmic solution groups.

### Method

TBUT measurement tests were conducted for 10 normal male cynomolgus monkeys by partial crossover design of 5 monkeys per group. The test animals were given general anesthesia by intramuscular injection with ketamine hydrochloride (Ketalar for intramuscular injection 500 mg (registered trademark), Daiichi Sankyo Company, Limited.), and then were let their eyes closed. Then, TBUT was measured before and at 15, 30, and 60 minutes after ocular instillation of a vehicle or each ophthalmic solution (20 μL/eye) to the right eye. Specifically, 1% fluorescein solution (2 μL/eye) was instilled, and the time right after opening of the eyelids until breaking of the fluorescein layer (tear film) on the cornea was measured three times under a slit lamp, followed by calculation of the average. (N=5 eyes)

### Results

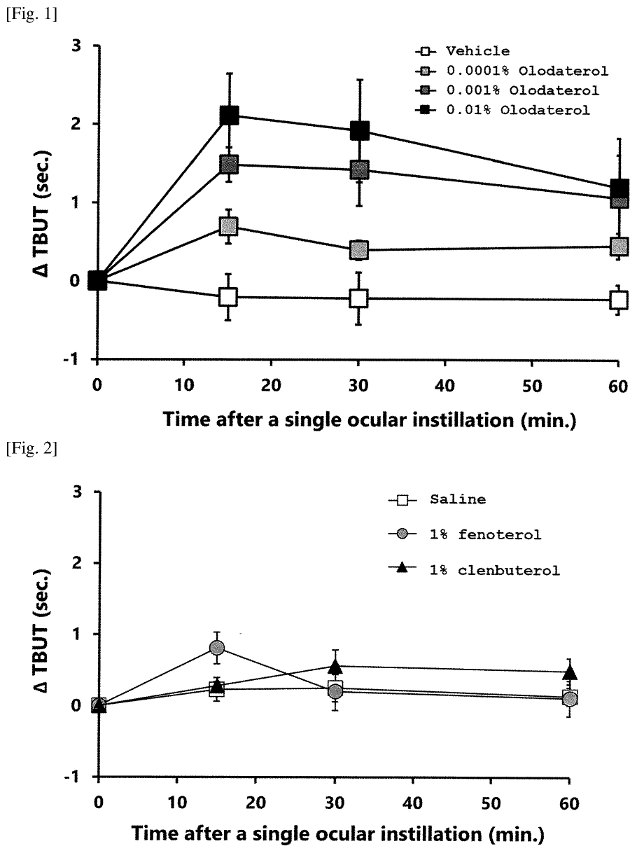

Test results are shown in FIG. 1. The ΔTBUT in FIG. 1 shows the difference between the TBUT averages of the olodaterol ophthalmic solution group for each concentration and the ophthalmic solution without containing olodaterol (vehicle) group (“control group”).

### Discussion

As shown in FIG. 1, the TBUT-extending effect was confirmed in a concentration-dependent manner in the ocular instillation of olodaterol. The TBUT-extending effect was not observed in ocular instillation of a vehicle.

Accordingly, olodaterol has the remarkable effect of stabilizing tear film, and is useful for a medicament for preventing and/or treating dry eye. In particular, it was indicated to be useful for a medicament for preventing and/or treating short tear film break-up time type of dry eye.

### [Test 2] Effect of Extending Tear Film Break-Up Time

The effects of fenoterol and clenbuterol ophthalmic solutions on extension of tear film break-up time were studied.

### Sample Preparation

The following ophthalmic solution 2 and ophthalmic solution 3 were prepared as a fenoterol ophthalmic solution and clenbuterol ophthalmic solution, respectively, for use in the test.

### Ophthalmic Solution 2: (1%)

Fenoterol hydrobromide (1 mg) was dissolved in physiological saline and water so as to be 100 μL, and a pH adjuster was added to the solution to prepare a fenoterol ophthalmic solution in the concentration of 1% (w/v).

### Ophthalmic Solution 3: (1%)

Clenbuterol hydrochloride (1 mg) was dissolved in physiological saline and water so as to be 100 μL, and a pH adjuster was added to the solution to prepare a clenbuterol ophthalmic solution in the concentration of 1% (w/v).

### Method

TBUT was measured in a similar manner to the method of Test 1. (N=4 or 5 eyes)

### Results

Test results are shown in FIG. 2. The ΔTBUT in FIG. 2 shows the difference between the TBUT averages of the fenoterol ophthalmic solution group or clenbuterol ophthalmic solution group and the physiological saline group without containing fenoterol and clenbuterol.

### Discussion

As shown in FIG. 2, the extension of TBUT was not confirmed in the fenoterol ophthalmic solution and clenbuterol ophthalmic solution, and the effect of stabilizing tear film was not observed. Whereas, as shown in FIG. 1, olodaterol, a selective β₂ adrenergic receptor agonist likewise, was confirmed to show the effect of stabilizing tear film even in a low concentration of 0.0001%. It was indicated that the effect of stabilizing tear film shown in olodaterol was not common to selective β₂ adrenergic receptor agonists, but was characteristic of olodaterol.

Accordingly, olodaterol has the unique and remarkable effect of stabilizing tear film, and is useful for a medicament for preventing and/or treating dry eye. In particular, it is expected to be useful for a medicament for preventing and/or treating short tear film break-up time type of dry eye.

### [Test 3] Effect of Increasing Tear Volumes

The effect of olodaterol ophthalmic solutions on increase of the quantity of tears was studied.

### Sample Preparation

The following ophthalmic solution 4 was prepared as an olodaterol ophthalmic solution for use in the test.

### Ophthalmic Solution 4:

Olodaterol hydrochloride (0.00001 g), a buffering agent, and a tonicity agent were dissolved in water so as to be 100 mL, and a pH adjuster was added to the solution to prepare an olodaterol ophthalmic solution in the concentration of 0.00001% (w/v).

Olodaterol ophthalmic solutions in the concentrations of 0.0001, 0.001, and 0.01% (w/v) were also prepared with varied amounts of olodaterol hydrochloride.

An ophthalmic solution without containing olodaterol (vehicle) was prepared as a control group for the olodaterol ophthalmic solution groups.

### Method

### Preparation of Dry-Eye Model

A somnopentyl injection five-fold diluted with physiological saline was intraperitoneally administered to a normal male SD (Sprague-Dawley) rat by 2.5 mL/kg, and the rat was given general anesthesia, followed by keeping the anesthetized state by inhalation of about 1 to 3% isoflurane. Then, the skin on each check was incised to remove the exorbital lacrimal gland, followed by suturation with a Michel suture clamp. In this test, an individual wherein reduction of tears was confirmed after 13 weeks from the removal of exorbital lacrimal gland was included as the experimental animal.

### Measurement of Quantity of Tears

The quantity of tears was measured with Schirmer strip before and at a prescribed time after ocular instillation of a vehicle or each ophthalmic solution. Specifically, pentobarbital was intraperitoneally administered right after (0 hour), 1.5 hours, 7.5 hours, and 15.5 hours after ocular instillation of a vehicle or each ophthalmic solution to both eyes (5 μL/eye). Half an hour after treatment of general anesthesia, Schirmer strip (1 mm×17 mm) was inserted to conjunctival sac of the lower eyelid of a rat, and then after 1 minute of the insertion, the wet part of the Schirmer strip was measured in increments of 0.5 mm. (N=14 or 16 eyes)

### Results

Test results are shown in FIG. 3.

### Discussion

As shown in FIG. 3, lacrimal secretion was enhanced in the olodaterol-instilled groups, and in the 0.0001 to 0.01% olodaterol-instilled groups, lacrimal secretion was highly enhanced even at 8 hours after ocular instillation. In the higher-concentration groups, the 0.001 and 0.01% olodaterol-instilled groups, the increase of tear volumes was observed even at 16 hours after ocular instillation, which showed the sustained effect of enhancing lacrimal secretion over a long time. Accordingly, olodaterol is useful for a medicament for preventing and/or treating dry eye, and in particular, it is expected to be useful for a medicament for preventing and/or treating aqueous deficient dry eye.

### [Test 4] Effect of Increasing Tear Volumes

The effects of fenoterol and clenbuterol ophthalmic solutions on increase of the quantity of tears was studied.

### Sample Preparation

The following ophthalmic solution 5 and ophthalmic solution 6 were prepared as a fenoterol ophthalmic solution and clenbuterol ophthalmic solution, respectively, for use in the test.

### Ophthalmic Solution 5:

Fenoterol hydrobromide (0.0001 g), a buffering agent, and a tonicity agent were dissolved in water so as to be 100 mL, and a pH adjuster was added to the solution to prepare a fenoterol ophthalmic solution in the concentration of 0.001% (w/v).

Fenoterol ophthalmic solutions in the concentrations of 0.01, 0.1, and 1% (w/v) were also prepared with varied amounts of fenoterol hydrobromide.

### Ophthalmic Solution 6:

Clenbuterol hydrochloride (0.0001 g), a buffering agent, and a tonicity agent were dissolved in water so as to be 100 mL, and a pH adjuster was added to the solution to prepare a clenbuterol ophthalmic solution in the concentration of 0.001% (w/v).

Clenbuterol ophthalmic solutions in the concentrations of 0.01, 0.1, and 1% (w/v) were also prepared with varied amounts of clenbuterol hydrochloride.

An ophthalmic solution without containing fenoterol or clenbuterol (vehicle) was prepared as a control group for the fenoterol or clenbuterol ophthalmic solution groups.

### Method

The quantity of tears was measured in a similar manner to Test 3. (N=10 eyes)

### Results

The following Tables 1 and 2 show incremental differences (mm/min) in Schirmer's test between the vehicle-instilled group and the ophthalmic-solution-instilled groups at 0.5 and 8 hours, respectively, after ocular instillation according to the results of Test 3 and Test 4. The symbol “-” in Tables 1 and 2 means not-performed.

TABLE 1
Incremental difference in Schirmer's test
at 0.5 hours after ocular instillation

| | 0.00001% | 0.0001% | 0.001% | 0.01% | 0.1% | 1% |
|---|---|---|---|---|---|---|
| olodaterol | 2.8 | 6.0 | 7.2 | 6.9 | — | — |
| fenoterol | — | — | 1.8 | 4.8 | 6.1 | 3.6 |
| clenbuterol | — | — | 2.0 | 2.7 | 3.6 | 3.0 |

TABLE 2
Incremental difference in Schirmer's
test at 8 hours after ocular instillation

| | 0.00001% | 0.0001% | 0.001% | 0.01% | 0.1% | 1% |
|---|---|---|---|---|---|---|
| olodaterol | 0.4 | 1.7 | 2.1 | 2.8 | — | — |
| fenoterol | — | — | 0.0 | 0.2 | 0.9 | 1.1 |
| clenbuterol | — | — | 0.3 | 1.1 | 2.4 | 3.9 |

### Discussion

According to the results in Table 1, olodaterol enhanced lacrimal secretion in much lower concentrations than fenoterol and clenbuterol. As shown in the results in Table 2, it was indicated that olodaterol has the remarkable and sustained effect even in a low concentration.

Accordingly, olodaterol has the remarkable effect of enhancing lacrimal secretion, and is useful for a medicament for preventing and/or treating dry eye. In particular, it is expected to be useful for a medicament for preventing and/or treating aqueous deficient dry eye.

### [Test 5] Intraocular Pressure Test

The effect of an olodaterol ophthalmic solution on intraocular pressure was studied.

### Sample Preparation

Ophthalmic solutions 1 (0.01 and 0.1%) were prepared as an olodaterol ophthalmic solution in a similar manner to Test 1 for use in the test. An ophthalmic solution without containing olodaterol (vehicle) was prepared as a control group for the olodaterol ophthalmic solution groups.

### Method

Intraocular-pressure measurement tests were conducted for 9 normal male cynomolgus monkeys by partial crossover design of 3 monkeys per group. A vehicle or each ophthalmic solution (20 μL/eye) was instilled to the right eye of the test animals twice daily repeatedly, and the intraocular pressure was measured right before and at 2 hours after the ocular instillation on Day 1, 7, and 14 and at 4 hours after the ocular instillation on Day 1. Specifically, one drop of 0.4% oxybuprocaine hydrochloride ophthalmic solution (trade name: Benoxil (registered trademark) ophthalmic solution 0.4%) was instilled to the right eye so as to provide surface anesthesia, and then the intraocular pressure was measured three times with Mentor tonometer (applanation), followed by calculation of the average. (N=6 eyes)

### Results

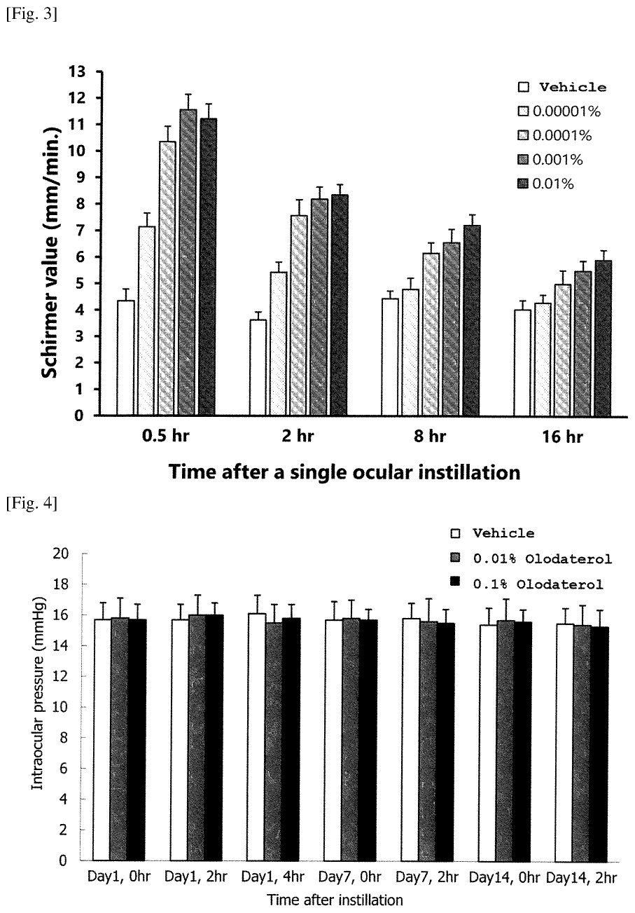

Test results are shown in FIG. 4.

### Discussion

As shown in FIG. 4, 0.01% and 0.1% olodaterol ophthalmic solutions did not show increase and/or decrease the intraocular pressure. Accordingly, olodaterol is expected to be a medicament without causing side effects that impact on the intraocular pressure.

### [Test 6] Effect of Extending Tear Film Break-Up Time

The effect of an olodaterol ophthalmic solution on extension of tear film break-up time was studied using a dry-eye model rat whose exorbital lacrimal gland is excised.

### Sample Preparation

The following ophthalmic solution 7 was prepared as an olodaterol ophthalmic solution for use in the test.

### Ophthalmic Solution 7:

Olodaterol hydrochloride (0.00001 g), a buffering agent, and a tonicity agent were dissolved in water so as to be 100 mL, and a pH adjuster was added to the solution to prepare an olodaterol ophthalmic solution in the concentration of 0.00001% (w/v).

Olodaterol ophthalmic solutions in the concentrations of 0.0001, 0.001, and 0.01% (w/v) were also prepared with varied amounts of olodaterol hydrochloride.

An ophthalmic solution without containing olodaterol (vehicle) was prepared as a control group for the olodaterol ophthalmic solution groups.

### Preparation of Dry-Eye Model Rat Whose Exorbital Lacrimal Gland is Excised

A male SD rat was given general anesthesia, the skin on each ear cheek was incised under the anesthetized state, and then the exorbital lacrimal gland was excised to prepare a dry-eye model rat whose exorbital lacrimal gland is excised.

### Method

Untreated normal rats were assigned to an untreated group, and dry-eye model rats whose exorbital lacrimal gland is excised were assigned to a vehicle group and each ophthalmic solution group, each group consisting of 6 rats. For each group, TBUT measurement tests were conducted.

TBUT was measured at 0.5 and 2 hours after ocular instillation of a vehicle or each ophthalmic solution (5 μL/eye) to both eyes of the rats. Specifically, half an hour after treatment of general anesthesia, FLUORES Ocular Examination Test Paper was inserted to each conjunctival sac of both eyes of the test animals to stain the surface of the eyes, and the time right after opening of the eyelids until breaking of the fluorescein layer (tear film) on the cornea was measured three times under a slit lamp, followed by calculation of the average. (N=12 eyes)

### Results

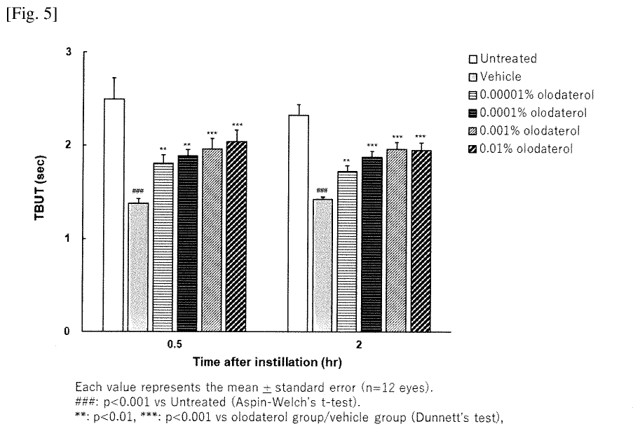

Test results are shown in FIG. 5. The TBUT in FIG. 5 shows the averages of the measured values of the olodaterol ophthalmic solution group for each concentration, the ophthalmic solution without containing olodaterol (vehicle) group (“control group”) and the untreated group.

### Discussion

As shown in FIG. 5, the TBUT-extending effect was confirmed at 0.5 and 2 hours after ocular instillation for the olodaterol ophthalmic solutions in the concentration of 0.00001% or more.

Accordingly, olodaterol has the remarkable and sustained effect of stabilizing tear film, and is useful for a medicament for preventing and/or treating dry eye. In particular, it was indicated to be useful for a medicament for preventing and/or treating short tear film break-up time type of dry eye.

### INDUSTRIAL APPLICABILITY

Olodaterol or a salt thereof may stabilize tear film and sustainably increase tear volumes even in a low concentration, and is expected to be useful for the prevention and/or treatment of dry eye.

1. A medicament for preventing and/or treating dry eye, comprising olodaterol or a salt thereof as an active ingredient.

2. The medicament according to claim 1, wherein the dry eye accompanies corneal and conjunctival epithelium disorder caused by at least one selected from the group consisting of superficial punctate keratopathy, corneal epithelium defect, corneal erosion, corneal ulcer, conjunctival epithelium defect, keratoconjunctivitis sicca, superior limbic keratoconjunctivitis, filamentary keratoconjunctivitis, non-infectious keratitis, and non-infectious conjunctivitis.

3. The medicament according to claim 1, wherein the dry eye is a disease caused by at least one selected from the group consisting of tear film instability, lacrimal hyposecretion, xerophthalmia, tear deficiency, keratoconjunctivitis sicca, lacrimal gland dysfunction, meibomian gland dysfunction, VDT (Visual Display Terminal) operation, use of contact lenses, tear abnormalities, and keratoconjunctivitis.

4. The medicament according to any one of claims 1 to 3, wherein the olodaterol or a salt thereof is comprised in a concentration of 0.1% (w/v) or less.

5. The medicament according to any one of claims 1 to 3, wherein the olodaterol or a salt thereof is comprised in a concentration of 0.01% (w/v) or less.

6. The medicament according to any one of claims 1 to 3, wherein the olodaterol or a salt thereof is comprised in a concentration of 0.00001 to 0.1% (w/v).

7. The medicament according to any one of claims 1 to 3, wherein the olodaterol or a salt thereof is comprised in a concentration of 0.00001 to 0.01% (w/v).

8. The medicament according to any one of claims 1 to 3, wherein the olodaterol or a salt thereof is comprised in a concentration of 0.00001 to 0.005% (w/v).

9. The medicament according to any one of claims 1 to 3, wherein the olodaterol or a salt thereof is comprised in a concentration of 0.00001 to 0.001% (w/v).

10. The medicament according to any one of claims 1 to 9, wherein the medicament is in the form of an eye drop.

11. The medicament according to any one of claims 1 to 10, wherein the medicament does not substantially increase and/or reduce intraocular pressure.

12. The medicament according to any one of claims 1 to 11, wherein the olodaterol or a salt thereof is olodaterol hydrochloride.

13. An agent for stabilizing tear film, comprising olodaterol or a salt thereof as an active ingredient.

14. An agent for enhancing lacrimal secretion and/or increasing tear volumes, comprising olodaterol or a salt thereof as an active ingredient.

15. Use of olodaterol or a salt thereof in the manufacture of a medicament for preventing and/or treating dry eye.

16. The use according to claim 15, wherein the dry eye accompanies corneal and conjunctival epithelium disorder caused by at least one selected from the group consisting of superficial punctate keratopathy, corneal epithelium defect, corneal erosion, corneal ulcer, conjunctival epithelium defect, keratoconjunctivitis sicca, superior limbic keratoconjunctivitis, filamentary keratoconjunctivitis, non-infectious keratitis, and non-infectious conjunctivitis.

17. The use according to claim 15, wherein the dry eye is a disease caused by at least one selected from the group consisting of tear film instability, lacrimal hyposecretion, xerophthalmia, tear deficiency, keratoconjunctivitis sicca, lacrimal gland dysfunction, meibomian gland dysfunction, VDT (Visual Display Terminal) operation, use of contact lenses, tear abnormalities, and keratoconjunctivitis.

18. The use according to any one of claims 15 to 17, wherein the concentration of the olodaterol or a salt thereof is 0.1% (w/v) or less.

19. The use according to any one of claims 15 to 17, wherein the concentration of the olodaterol or a salt thereof is 0.01% (w/v) or less.

20. The use according to any one of claims 15 to 17, wherein the concentration of the olodaterol or a salt thereof is 0.00001 to 0.1% (w/v).

21. The use according to any one of claims 15 to 17, wherein the concentration of the olodaterol or a salt thereof is 0.00001 to 0.01% (w/v).

22. The use according to any one of claims 15 to 17, wherein the concentration of the olodaterol or a salt thereof is 0.00001 to 0.005% (w/v).

23. The use according to any one of claims 15 to 17, wherein the concentration of the olodaterol or a salt thereof is 0.00001 to 0.001% (w/v).

24. The use according to any one of claims 15 to 23, wherein the medicament is in the form of an eye drop.

25. The use according to any one of claims 15 to 24, wherein the medicament does not substantially increase and/or reduce intraocular pressure.

26. The use according to any one of claims 15 to 25, wherein the olodaterol or a salt thereof is olodaterol hydrochloride.

27. Use of olodaterol or a salt thereof in the manufacture of a medicament for stabilizing tear film.

28. Use of olodaterol or a salt thereof in the manufacture of a medicament for enhancing lacrimal secretion and/or increasing tear volumes.
